(19) United States
(12) Patent Application Publication
(10) Pub. No.: US 2024/0041648 A1
(43) Pub. Date: Feb. 8, 2024

(54) EYE DROP MONITORING DEVICE AND METHOD

(71) Applicant: TWENTY TWENTY THERAPEUTICS LLC, South San Francisco, CA (US)

(72) Inventors: Supriyo Sinha, Menlo, CA (US); Fred Lee, South San Francisco, CA (US); Oleg Rumyantsev, Saint Johns, FL (US); Hojr Sedaghat Pisheh, Pinole, CA (US)

(21) Appl. No.: 18/229,573

(22) Filed: Aug. 2, 2023

Related U.S. Application Data
(60) Provisional application No. 63/394,324, filed on Aug. 2, 2022.

(51) Int. Cl. A61F 9/00

(52) U.S. Cl. CPC A61F 9/0026

(57) ABSTRACT

An eye drop monitoring device for monitoring gravity-driven placement of an eye drop into an eye of a patient includes a light source configured to selectively direct light toward at least one of the eye and the eye drop. The light is backscattered by at least one of the eye and the eye drop. A light detector is configured to detect the backscattered light from at least one of the eye and the eye drop and responsively produce at least one backscatter signal. An accelerometer is configured to determine a spatial position of the device and responsively produce a device position signal. A processing unit is configured to receive the at least one backscatter signal and the device position signal and responsively produce a drop-on-target signal indicative of a likelihood that the eye drop fell into a predetermined position with respect to the eye.

### RELATED APPLICATION

This application claims priority from U.S. Provisional Application No. 63/394,324, filed 2 Aug. 2022, the subject matter of which is incorporated herein by reference in its entirety.

### TECHNICAL FIELD

This disclosure relates to an apparatus and method for use of an eye drop monitoring device and, more particularly, to a method and apparatus of an eye drop monitoring device for monitoring gravity-driven placement of an eye drop into an eye of a patient.

### BACKGROUND

It can be difficult for a patient to align an eye dropper bottle with her eyeball sufficiently to target the gravity-driven insertion of an eyedrop liquid precisely into the eye. Repeated unsuccessful attempts to self-administer the eyedrops can result in mess and wasted liquid, as well as patient discomfort and anxiety. This is an issue, both for the patient (since the medication is not reaching the intended target), and also for the medical provider (as they cannot assess if a conditioning is not improving because the dropper medication is not effective or because the user is not applying the drop correctly).

A camera with a suitably high frame rate can be used to determine if a droplet enters the eye, but the addition of an image sensor and the circuitry to process the images can increase the cost of the device dramatically. In addition, this circuitry can increase the power consumption of the device, which could result in an increase in the size of an onboard battery or decrease in the time between charges or battery replacements.

### SUMMARY

In an aspect, alone or in combination with any other aspect, an eye drop monitoring device for monitoring gravity-driven placement of an eye drop into an eye of a patient is described. The device comprises a light source configured to selectively direct light toward at least one of the eye and the eye drop. The light is backscattered by at least one of the eye and the eye drop. A light detector is configured to detect the backscattered light from at least one of the eye and the eye drop and responsively produce at least one backscatter signal. An accelerometer is configured to determine a spatial position of the device and responsively produce a device position signal. A processing unit is configured to receive the at least one backscatter signal and the device position signal and responsively produce a drop-on-target signal indicative of a likelihood that the eye drop fell into a predetermined position with respect to the eye

### BRIEF DESCRIPTION OF THE DRAWINGS

For a better understanding, reference may be made to the accompanying drawings, in which:

FIG. 1 is a schematic side view of an eye dropper bottle and an eye drop monitoring device; and

FIG. 2 is a schematic view of a light backscattering plot related to operation of the eye drop monitoring device of FIG. 1.

### DESCRIPTION OF ASPECTS OF THE DISCLOSURE

Unless defined otherwise, all technical and scientific terms used herein have the same meaning as is commonly understood by one of skill in the art to which the present disclosure pertains.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “directly adjacent” another feature may have portions that overlap or underlie the adjacent feature, whereas a structure or feature that is disposed “adjacent” another feature might not have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of a device in use or operation, in addition to the orientation depicted in the figures. For example, if a device in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

The invention comprises, consists of, or consists essentially of the following features, in any combination.

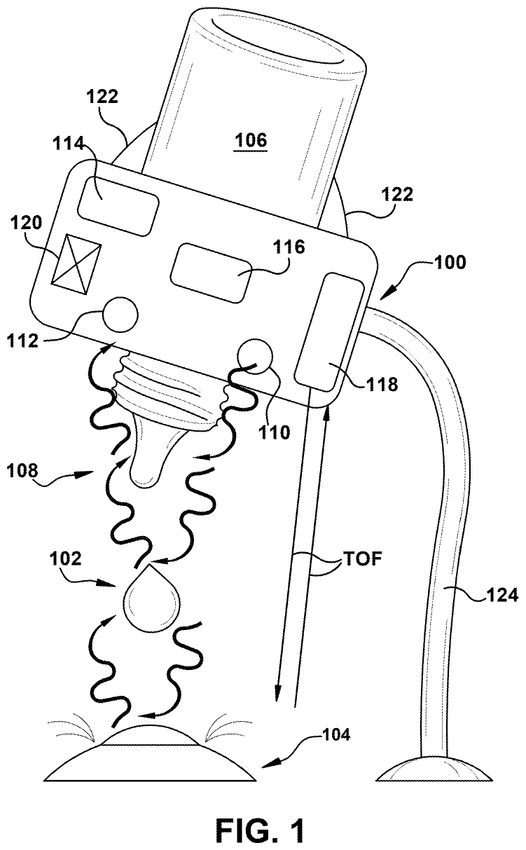

FIG. 1 depicts a device 100 for monitoring administration, via gravity-driven placement, of an eye drop 102 to an eye 104 of a patient. The eye drop monitor device 100 can be used in conjunction with an eye drop container 106 or other container, supplied separately from or concurrently with the device 100. The eye drop container 106 includes a nozzle 108, which can be configured to deliver selected fluid(s) to the eye, in the form of drops, mist, micro-sheet, or any other form as desired. Although a single nozzle 108 is shown, the eye drop container 106 could include multiple nozzles.

The eye drop monitoring device 100 may be used to assist with determining whether an eye drop 102 actually fell into the patient's eye 104 (at a time when the eye is open [non-blinking]). The device 100 can be a real-time indicator (binary yes/no indication of whether the eye drop 102 was likely administered to the patient's eye 104), and/or can store historical information, such as a log of the administration attempts and successes/results. When a storage feature is provided, such storage and display/access can be done locally on the device 100 and/or via a wired or wireless connection to an external device, such as, but not limited to, a smartphone and/or a device storing an electronic medical record for the patient. When external device storage/access is provided, the indication of an eye drop 102 administration event/results could be stored for later “pull” access and/or could be “pushed” to a medical provider, caregiver, insurer, or any other desired party.

It is contemplated that the device 100 could be integrated into an eye drop container 106, or could instead be provided separately and attached to the bottle 106 as desired (as needed or at least semi-permanently), as will be discussed below. For some use environments of the device 100, it may be important to the internal algorithms/logic for a distance between the device (or certain components thereof) and a drop-emitting portion of the nozzle 108 to be known; in these cases, a robust and substantially immobile affixation of the device 100 to the eye drop container 106 may be appropriate.

The user (patient or caregiver) squeezes or otherwise actuates the eye drop container 106 in a known manner to release the eye drop 102 at a time of her own choosing, in the below description. However, it is contemplated that an automated release (timed, on-demand, responsive to the device 100 determination, or in response to any other stimulus) of the eye drop 102 from an appropriately configured eye drop container 106 could be also or instead available.

The device 100 may include a display configured to present, in a user-perceptible format, salient information such as, but not limited to, number of doses/drops 102 administered, number of doses/drops 102 remaining in the bottle 106, expiration date of the drops 102, time elapsed since a previous event (e.g., initial access to the bottle 106, last successful administration), time remaining until a future event (e.g., expiration date of the bottle 106, next medical appointment pertaining to the drops 102, next planned administration), and/or any other information which may be helpful to a user of the device 100. A “user-perceptible” display may include visually perceptible information (text, one or more lights, one or more legible symbol-containing displays such as an LCD, a dial or graph, or any other desired visible indicator), auditorily perceptible information (recorded message, tone, music, buzzing sound, or any other desired audible information), tactilely perceptible information (vibration, change of temperature, or any other desired haptic information), and/or any other indication which can be perceived by an intended user.

With reference once again to FIG. 1, the device 100 includes a light source 110 configured to selectively direct light toward at least one of the eye 104 and the eye drop 102. The light source may emit light in the visible spectrum, the infrared portion of the spectrum, the ultraviolet portion of the spectrum, and/or any light or other electromagnetic radiation (considered “light” for the purposes of this description) which can backscatter from a surface. The light source could include, for example, one or more LEDs, laser diodes, incandescent light sources, halogen light sources, mirrors, or any other desired light-emitting and/or -directing component.

The light emitted by the light source 110—represented by the wiggly downward-pointing arrows, in the orientation of FIG. 1—is backscattered by at least one of the eye 104 and the eye drop 102. A light detector 112 is configured to detect the backscattered light—represented by the wiggly upward-pointing arrows, in the orientation of FIG. 1—from at least one of the eye 104 and the eye drop 102 and responsively produce at least one backscatter signal. An open eye 104 (e.g., a cornea) is more reflective of light than is a patient's skin (e.g., an eyelid or skin surrounding the eye socket), and so the backscatter signal can be analyzed and compared to known and/or expected spatiotemporal values to determine whether the patient is blinking and/or whether the nozzle 108 is aligned as desired with the eye 104. Light backscattered from the eye drop 102 has a different spatial profile compared to that of light backscattered and/or reflected from the patient's eye 104, and so the backscatter signal can be analyzed to determine a position of the eye drop 102 in relation to the “background” placement of the eye 104. Actuation of the light source 110 and/or the detector 112 may be done manually by the user, responsive to a predetermined motion (e.g., inversion) of the bottle 106 (as will be discussed below), automatically at predetermined time intervals, or in response to any other desired actuation stimulation.

In some use environments, the light source 110 is a first light source, and the device 100 may include a second light source, of any suitable type, and similar to or different from the first light source. In this situation, a selected one of the first and second light sources 110 may direct light toward the eye 104 and the other one of the first and second light sources 110 may direct light toward the eye drop 102. In such a situation, the detector 112 may be a first detector, and the device 100 may include a second detector. A selected one of the first and second detectors 112 detects light backscattered from the eye 104 and the other one of the first and second detectors 112 detects light backscattered from the eye drop 102. It is contemplated, though, that any desired number, configuration, and location of light sources 110 may emit light toward the eye 104 and/or the eye drop 102, and that any desired number, configuration, and location of detectors 112 may detect the backscattered light from the eye drop 102, the eye 104, or any other surface relevant to the monitoring function of the device 100.

An accelerometer 114 is configured to determine a spatial position of the device 100 and responsively produce a device position signal. For example, one or more accelerometer 114 sensors can be used to detect pitch, yaw, and/or roll of the device 100, and/or a relative or absolute position of the device 100 in relation to a predetermined point of reference. The device position signal can include a temporal component, as another example, which can help indicate that the eye drop container 106 has likely been held in an inverted position a sufficient length of time that it is likely positioned above a patient's eye 104.

A processing unit 116 is configured to receive the at least one backscatter signal and the device position signal and responsively produce a drop-on-target signal indicative of a likelihood that the eye drop 102 fell into a predetermined position with respect to the eye 104. For example, the processing unit 116 may be configured to receive the at least one backscatter signal and determine whether the nozzle 108 is likely in position above the eye 104 (as opposed to “off target” on another portion of the face), potentially with reference to the device position signal to determine whether the bottle 106 is being held in a drop-administering posture.

The processing unit 116 may also or instead be configured, for example, to receive the at least one backscatter signal and responsively determine an open/closed condition of the eye 104 of the patient. (I.e., whether the patient has blinked within a predetermined period of time relative to release of the drop 102, which can be sensed via the backscatter signal(s), as well.) When such a determination is made, the processing unit 116 determines the drop-on-target signal at least partially responsive to the determination of the open/closed condition of the eye of the patient. The processing unit 116 may be configured to produce a user-perceptible non-blink indication responsive to the determination of the open/closed condition of the eye 104 of the patient, and/or responsive to the determination of the drop-on-target signal. For example, the processing unit 116 may control red/green LEDs on a surface of the device 100 facing the patient, so that the patient sees a red LED light when the bottle 106 is in an “off target” posture (e.g., pointing toward eye-adjacent skin and/or the eyelid of a partially closed eye 104, whether mid-blink or merely squinted). The green LED would then be activated (and the red LED turned off) when the backscattered light (and any other factors considered) indicates that a drop-on-target value is within a predetermined range of acceptable values, indicating likely successful administration of the eye drop 102 to the eye 104. It is contemplated that the processing unit 116 may be located remotely from one or more other components of the device 100—for example, the device 100 could provide the described sensing and signal generation functions, whereas a smartphone or other separate device could perform the duties of the described processing unit 116.

As mentioned previously, the processing unit 116 may include at least one non-transitory computer-readable media configured to selectively store at least one drop-on-target signal in an “onboard” manner. An onboard processing unit 116 may also or instead be in wired or wireless communication, frequently, occasionally, or rarely, with at least one external device including non-transitory computer-readable media configured to selectively store at least one drop-on-target signal (such as via an entry in a patient-specific electronic medical record. The external device can include various systems and subsystems. The external device can be a personal computer, a laptop computer, a workstation, a computer system, an appliance, an application-specific integrated circuit (ASIC), a server, a server blade center, a server farm, etc.

The external device can include a system bus, a system processing unit, a system memory, one or more memory devices, a communication interface (e.g., a network interface), a communication link, a display (e.g., a video screen), and an input device (e.g., a keyboard and/or a mouse). The system bus can be in communication with the system processing unit and the system memory. The additional memory device(s), such as a hard disk drive, server, stand alone database, or other non-volatile memory, can also be in communication with the system bus. The system bus interconnects the system processing unit, the memory device(s), the communication interface, the display, and the input device. In some examples, the system bus also interconnects an additional port, such as a universal serial bus (USB) port. Additionally or alternatively, the system can access an external data source or query source through the communication interface, which can communicate with the system bus and the communication link.

The device 100 may include plurality of detectors 112, each being configured to detect the backscattered light from at least one of the eye 104 and the eye drop 102 and responsively produce at least one backscatter signal. In this situation, the processing unit 116 selects at least one chosen backscatter signal (from all of the backscatter signals produced by the plurality of detectors 112) for use in producing the drop-on-target signal. This selection may be made responsive to the device position signal. For example, if the accelerometer 114 determines that the device 100 is tilted a certain direction, the processing unit 116 may “know” that a certain one of the plurality of detectors 112 is likely to be blocked by the nozzle 108 or otherwise likely to give a misleading or inaccurate signal. That certain detector 112 can be ignored by the processing unit 116, in favor of one or more other backscatter signals, when producing the drop-on-target signal.

The device 100 may include at least one proximity sensor 118 configured to detect a distance between the device 100 (or a predetermined portion thereof) and at least one of an eye 104 and an eye-adjacent portion of a face of the patient. This detection is represented schematically in FIG. 1 by time-of-flight arrows TOF. The proximity sensor 118 detects the distance and responsively produces a proximity signal. The processing unit 116 produces the drop-on-target signal at least partially responsive to the proximity signal. The proximity signal gives the processing unit 116 data which can be helpful in estimating how well the eye drop 102 is aligned with respect to the eye 104, especially when the device o]100 is tilted. The proximity signal may also be useful to help determine a time-of-flight of the eye drop 102, traveling under gravitational pull, to the eye 104. This “travel duration” may be helpful in, for example, setting a “non-blink” window of time during which the eye drop 102 is likely to have landed in an open eye, detecting certain backscatter characteristics of the eye drop 102 (see, e.g., chart “C” in the Appendix), “weighting” particular portions of the backscatter plot, or in any other desired task which can be aided by knowing how long the eye drop 102 is traveling. The “travel duration” calculation may be aided by knowledge of the distance between the drop-emitting apertured portion (“tip”) of the nozzle 108 and another component of the device 100 (e.g., the proximity sensor 118, when present), so that the time-of-flight can be offset to reflect the actual distance traveled by a falling eye drop 102.

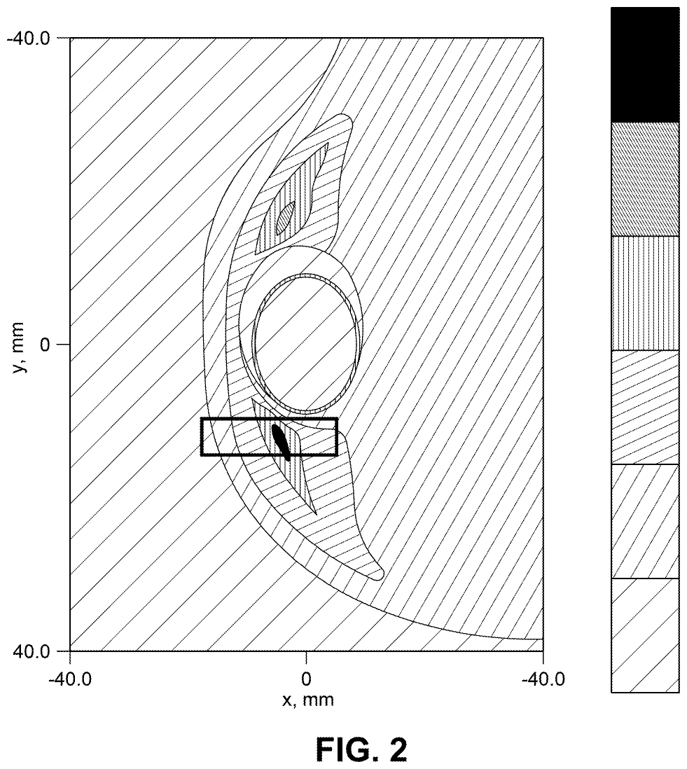

FIG. 2 depicts an example backscatter plot for one implementation of the device 100. The leftmost rectangle in FIG. 2 plots the values (mapped to color intensities as shown in the rightmost key column in that Figure) of backscattered light that are detected within a plane (which is substantially parallel to a plane tangent to the uppermost eye 104 surface). The plane is shown as being taken at a distance from the eye 104 corresponding to a position of the detector 112 (represented by the small white rectangle in the plot) upon the device 100. As is apparent from the plot, a “shadow” of the nozzle 108 appears in a central position as a low-intensity backscatter area. In the particular implementation of the device 100 mapped as FIG. 2, the location and configuration of the light source 110 result in backscattered light from the eye drop 102 which manifests as a shallow “C”-shaped area of brighter intensity. For the configuration of the device 100 associated with FIG. 2, and the tilt angle from which the drop 102 is falling, the brightest backscatter coincides with the location of the selected detector 112 represented by the white box. As a result, the location of the drop 102 relative to the components of the device 100 (e.g., before, during, and/or after impact on the eye 104) can be calculated. This calculation may take into account, for example, if the detector 112 has multiple individual “pixels”. Alternatively, a signal from a single pixel type detector 112 integrating the intensity of light hitting it may be used to detect the time of the application of the drop 102 based on the temporal profile of the detected signal.

A power source 120 is configured to provide electrical power to at least one of the light source(s), light detector(s), accelerometer(s), proximity sensor(s), and processing unit. The power source 120 may be of any desired type, and may be replaceable, rechargeable (in a wired or wireless manner), or permanently affixed to the device 100 and nonrechargeable. In this last situation, the power source 120 should be selected to provide a predetermined useful life span for the device 100 as desired for a particular use application.

Any suitable user-perceptible indication of the drop-on-target result could be provided to the user shortly after a drop 102 is administered to the eye 104 area. For example, a green LED could light if the drop-on-target signal value is within a predetermined range indicating likely success in drop 102 administration to an open eye at an appropriate distance and angle from the eye drop container 106. A red LED could light if the drop-on-target signal value indicates that a therapeutically effective volume of the drop 102 did not reach an open eye 104 successfully, so that the user can either note the failure and/or try again. (These indicator LEDs could be the same as, or different than, one or more light sources 110.) As suggested previously, this “drop-on-target” information can be saved—via the device or an external unit (e.g., smartphone)—for later review by a medical professional, caregiver, or other appropriate party. It is also contemplated that such an external party could be notified in real-time or shortly after the drop 102 administration, of the associated drop-on-target signal value.

As previously noted, it may be desirable to have a substantially fixed positioning of the device 100 with respect to the eye drop container 106, both for ease of using these two items together, as well as for potential computational advantages in having a known spatial relationship between the device 100 and the nozzle 108, or another feature, of the eye drop container 106. Accordingly, the device 100 may include a mounting feature (shown schematically at 122) for selectively attaching the device 100 to an eye drop container 106. This mounting feature 122 may include, but is not limited to, a resilient C-clamp for accepting the eye drop container 106, a tabbed washer through which the nozzle 108 is inserted and held, a threaded feature for engagement with a corresponding thread on the nozzle 108, an adhesive, an elastic/rubber/resilient band for gripping the eye drop container 106, a socket into which the eye drop container 106 may be at least partially inserted, a frictional fit feature, or any other desired mounting feature 122 or combination thereof. One of ordinary skill in the art can readily provide a suitable mounting feature for a particular use environment of the device 100. The mounting feature 122, when present, may removably attach the device 100 to the eye drop container 106. Conversely, the mounting feature 122, when present, may at least semi-permanently (i.e., without intent for removal during the remaining useful life cycle of the eye drop container 106) attach the device 100 to the eye drop container 106. It is contemplated that the device 100 could be integrally formed with the eye drop container 106, during manufacture thereof. It is also contemplated that a particular mounting feature 122 could be configured to only accept an eye drop container 106 having a predetermined “mating” shape, to avoid accidental administration to the eye of an unintended, and potentially harmful, substance (e.g., a contact lens cleaning solution).

The device 100 may include a holdoff feature (shown schematically at 124) for selectively contacting a face of the patient. When present, the holdoff feature 124 spaces the device 100 a predetermined distance from the eye 104. The holdoff feature 124 could be, for example, an eye cup configured as a “skirt” (or at least a portion thereof) which extends downward (in the orientation of FIG. 1) from the device 100 to at least partially surround the eye socket. When present, the holdoff feature 124 may supplement or supplant the proximity sensor 118 in aiding the processing unit 116 to determine a distance between the device 100 (or portions thereof) and the patient's eye 104.

As a further refinement of the holdoff feature 124, the device 100 may be configured to both selectively accept an eye drop container 106 and maintain the eye drop container 106 in a predetermined spatial relationship with respect to the eye 104. For example, the device 100, or a portion thereof, could be substantially shaped as a three-dimensional hourglass, with a bottom, bell-shaped portion functioning as an eye cup and a top, U-shaped portion accepting at least one configuration of eye drop container 106 to hold the nozzle 108 adjacent, or within, the “neck” of the hourglass.

It is contemplated that, for purposes of providing dimensions of the eye drop container 106 to the device 100 or for any other reason, a chosen eye drop container 106 could be indicated to the device 100 at the time the device 100 is associated with the eye drop container 106 or at any other suitable time. This identification could be accomplished manually or automatically, via a scheme such as, but not limited to, low frame rate camera, manual entry, separate camera (e.g., smartphone), RFID chip, mechanical interlock or indicator, any other identification means, or any combination thereof.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages can be obtained from a study of the drawings, the disclosure, and the appended claims.

We claim:

1. An eye drop monitoring device for monitoring gravity-driven placement of an eye drop into an eye of a patient, the device comprising:
a light source configured to selectively direct light toward at least one of the eye and the eye drop, the light being backscattered by at least one of the eye and the eye drop;
a light detector configured to detect the backscattered light from at least one of the eye and the eye drop and responsively produce at least one backscatter signal;
an accelerometer configured to determine a spatial position of the device and responsively produce a device position signal; and
a processing unit configured to receive the at least one backscatter signal and the device position signal and responsively produce a drop-on-target signal indicative of a likelihood that the eye drop fell into a predetermined position with respect to the eye.

2. The device of claim 1, wherein the light source is configured to emit light in the visible spectrum.

3. The device of claim 1, wherein the light source is a first light source and the device includes a second light source, wherein a selected one of the first and second light sources directs light toward the eye and the other one of the first and second light sources directs light toward the eye drop.

4. The device of claim 1, wherein the detector is a first detector and the device includes a second detector, wherein a selected one of the first and second detectors detects light backscattered from the eye and the other one of the first and second detectors detects light backscattered from the eye drop.

5. The device of claim 1, including a plurality of detectors, each being configured to detect the backscattered light from at least one of the eye and the eye drop and responsively produce at least one backscatter signal, and wherein the processing unit selects at least one chosen backscatter signal for use in producing the drop-on-target signal responsive to the device position signal.

6. The device of claim 1, including a proximity sensor configured to detect a distance between the device and at least one of an eye and an eye-adjacent portion of a face of the patient and responsively produce a proximity signal, and wherein the processing unit produces the drop-on-target signal at least partially responsive to the proximity signal.

7. The device of claim 1, wherein the processing unit is configured to receive the at least one backscatter signal and responsively determine an open/closed condition of the eye of the patient.

8. The device of claim 7, wherein the processing unit produces a user-perceptible non-blink indication responsive to the determination of the open/closed condition of the eye of the patient.

9. The device of claim 7, wherein the processing unit determines the drop-on-target signal at least partially responsive to the determination of the open/closed condition of the eye of the patient.

10. The device of claim 1, wherein the processing unit produces a user-perceptible on/off target indication responsive to the determination of the drop-on-target signal.

11. The device of claim 1, wherein the processing unit includes non-transitory computer-readable media configured to selectively store at least one drop-on-target signal.

12. The device of claim 1, wherein the processing unit is in communication with at least one external device including non-transitory computer-readable media configured to selectively store at least one drop-on-target signal.

13. The device of claim 1, including a power source configured to provide electrical power to at least one of the light source, light detector, accelerometer, and processing unit.

14. The device of claim 1, including a mounting feature for selectively attaching the device to an eye drop container.

15. The device of claim 14, wherein the mounting feature removably attaches the device to the eye drop container.

16. The device of claim 1, including a holdoff feature for selectively contacting a face of the patient, the holdoff feature spacing the device a predetermined distance from the eye.

17. The device of claim 1, wherein the device is configured to selectively accept an eye drop container and maintain the eye drop container in a predetermined spatial relationship with respect to the eye.

18. A method of monitoring gravity-driven placement of an eye drop into an eye of a patient, the method comprising:
providing the device of claim 1;
placing the device above the eye of the patient;
with the light source, directing light toward at least one of the eye and the eye drop, the light being backscattered by at least one of the eye and the eye drop;
with the light detector, detecting the backscattered light from at least one of the eye and the eye drop and responsively producing at least one backscatter signal;
with the accelerometer, determining a spatial position of the device and responsively producing a device position signal;
transmitting the at least one backscatter signal and the device position signal to the processing unit;
producing a drop-on-target signal responsive to the transmitted at least one backscatter signal and the device position signal; and
with the drop-on-target signal, indicating a likelihood that the eye drop fell into a predetermined position with respect to the eye.
